(19) United States
(12) Patent Application Publication
(10) Pub. No.: US 2011/0082382 A1
(43) Pub. Date: Apr. 7, 2011

(54) BIOELECTRICAL IMPEDANCE MEASURING APPARATUS

(75) Inventor: Frank Willers, Lubeck (DE)

(73) Assignee: SECA AG, Reinach BL 1 (CH)

(21) Appl. No.: 12/896,545

(22) Filed: Oct. 1, 2010

(30) Foreign Application Priority Data
Oct. 1, 2009 (EP) 09171974.0

(51) Int. Cl. A61B 5/053

(52) U.S. Cl. 600/547

(57) ABSTRACT

The present invention provides a bioelectrical impedance measuring apparatus for determining composition data of a human body, the apparatus including several measuring electrodes, measuring circuitry on a main board including voltage measuring circuitry and a control and analysis unit. The control and analysis unit is arranged to apply, according to a predetermined measuring program, an alternating current from a controllable alternating current source to an electrode specific for the respective measuring program to the body and to conduct away alternating current via another electrode and to determine by means of two further electrodes with the voltage measuring circuitry the resulting voltage, and to determine on this basis the impedance of a body segment. Each voltage applying electrode includes a remotely controlled alternating current source separate from the main board and disposed in the vicinity of the current applying electrode.

### CROSS-REFERENCE TO RELATED APPLICATION

The present application claims the benefit of and priority from European Patent Application Serial No. EP 09171974.0, filed Oct. 1, 2009, the entire disclosure of which is hereby incorporated by reference herein.

### BACKGROUND OF THE INVENTION

1. Field of the Invention

The present invention relates to a bioelectrical impedance measuring apparatus for determining composition data of the human body.

2. Discussion of the Prior Art

The electrical conductivity of a human body is strongly influenced by its water content. Since areas of the body which are free of fat, such as muscles and bodily fluids contain the major part of the water content of the body, while on the other hand fat tissue has a relatively low water content, the determination of the conductivity of a body or of a body segment (or the determination of the reciprocal resistance or impedance of the body or of the body segment) allows to draw conclusions on the relative content of fat, at least if further data such as body height and weight of the person are taken into account.

Typical bioelectrical impedance measuring apparatus comprise eight electrodes, namely four foot electrodes, in each case two for contacting one foot, and four hand electrodes, in each case two for contacting one hand of the person. For each of the limbs one electrode can be assigned for applying or injecting current. An alternating current is injected through one electrode and is conducted away through another electrode which is located on another limb, and using two further electrodes, which are likewise positioned on different limbs, the voltage is measured. By turning to other pairs of electrodes for current injection and conducting away and for sensing voltage differences different body segments may be examined consecutively. When current is injected in one hand and in one foot and when the voltage is measured on the other electrode on the same hand and on the other electrode of the same foot, one whole side of a body may be measured.

Most measuring circuits as well as the control and analysis unit are located on a main board which is connected by cables with the remotely positioned electrodes at the limbs. In particular, also the sources for an alternating current which generate an alternating current with an amplitude controlled by the control and analysis unit for applying it to the body are located on the main board. Such a circuit design is schematically shown in FIG. 3. On the main board 1 the control and analysis unit 2 is located which is connected to a voltage measuring circuit 10 which in turn is connected to electrodes 16 and 17. The control and analysis unit 2 also receives the output signal of a current measuring circuit 11. In addition, the control and analysis unit 2 supplies a control signal by which the amplitude of the alternating current source 3 is determined. From the alternating current source 3 alternating current is applied via a cable 19 and further via the electrode 15 into the body. In this arrangement current is applied through the cables 19 and 22, and voltage is measured between the electrodes 16 and 17. For a precise measurement of the impedance 14 the current applied to the body has to be known as precisely as possible. In this respect the set-up shown in FIG. 3 is disadvantageous since the alternating current generated by the alternating current source 3 has to be conducted over a fairly long conductor to the point of application at the electrode at the body. Parasitic capacities of the conductor result in losses, in particular for higher frequency alternating currents, so that the actually injected alternating current is not precisely known. As a result, also the impedance can not be determined in a precise manner.

A known apparatus for bioelectrical impedance analysis is described in WO 97/01303. The schematical set-up of the circuits is shown in FIG. 4 which, for most parts, corresponds to the set-up described above in connection with FIG. 3, except that the preamplifiers 6 and 8 are separated from the main board 1 and positioned closer to the voltage measuring electrodes, which preamplifiers transmit the output signals to the voltage measuring circuit 10. Furthermore, a current measuring circuit 12 is disposed separately from the main board, by which current measuring circuit 12 the current conducted away through the electrode 18 is determined, and the determined value is transmitted to the control and analysis unit 2. Also in this arrangement the alternating current to be applied is generated by an alternating current source 3 on the main board and suffers during the further conduction to the applying electrode 15 losses by parasitic capacitances, which losses can not be precisely predicted. In this arrangement, however, the actually flowing current is measured by the current measuring circuit 12 so that this measured current can be taken into account together with the voltage measured between the electrodes 16 and 17 in order to determine the impedance. However, in this arrangement an additional current measuring circuit 12 is needed.

### SUMMARY

It is an object of the present invention to provide a bioelectrical impedance measuring apparatus having a simple set-up by which the impedance maybe determined a precise manner; in particular this is to be achieved by a precise adjustment of the applied current.

According to the invention each current applying electrode is provided with an alternating current source which is separate from the main board and disposed in the vicinity of the current applying electrode and which is under remote control of the control and analysis unit. In this manner an alternating current having the desired amplitude is generated close to the current applying electrode and then supplied to this electrode. In this way the amplitude of the applied alternating current is well known and is not affected by parasitic capacitances on the way to the electrode. From the control and analysis unit on the main board merely control signals have to be transmitted to each current source, which control signals prescribe the amplitude of the alternating current to be generated. Such control signals can be transmitted from the control and analysis unit to the remote electrodes in a manner which is less susceptible to interferences so that there is no uncertainty regarding the actually generated amplitude of the alternating current. With the precisely known amplitude of the applied alternating current at the electrode, the voltage between two further electrodes may simply be measured, and based on the known amplitude and measured voltage the impedance may be determined eventually, without need for a separate current measurement of the actually applied alternating current or of the alternating current conducted away. The current measuring circuitry is thus dispensable. In this manner a set-up having a simple circuit design is realized which nevertheless allows measurement of the impedance with high accuracy.

In a preferred embodiment the preamplifiers for the voltage measuring electrodes are likewise disposed in the vicinity of the respective electrodes in order to minimize in this manner the capacitance due to the current path before the preamplifiers inputs.

In a preferred embodiment the remote control of the alternating current source is performed by the control and analysis unit using a differential signal which is transmitted via a twisted pair cable. Alternatively, the control of the alternating current source can be carried out using a signal which is transmitted to the alternating current source via shielded conductor.

In a preferred embodiment the voltage measuring signals from the preamplifiers disposed close to the voltage measuring electrodes maybe transmitted as differential signals over twisted pair cables to the control and analysis unit, or may be transmitted as absolute signals over shielded cables to the control and analysis unit. Furthermore, in a preferred embodiment each electrode conducting current away may be connected to the control analysis unit via a shielded cable.

This summary is provided to introduce a selection of concepts in a simplified form that are further described below in the detailed description of the preferred embodiments. This summary is not intended to identify key features or essential features of the claimed subject matter, nor is it intended to be used to limit the scope of the claimed subject matter.

Various other aspects and advantages of the present invention will be apparent from the following detailed description of the preferred embodiments and the accompanying drawing figures.

### BRIEF DESCRIPTION OF THE DRAWING FIGURES

The invention will be in the following described with reference to embodiments illustrated in the drawings in which:

FIG. 1 shows a schematical circuit diagram of a bioelectrical impedance measuring apparatus,

FIG. 2 shows a schematical circuit diagram of an alternative embodiment of a bioelectrical impedance measuring apparatus,

FIG. 3 shows a schematical circuit diagram of a bioelectrical impedance measuring apparatus of the prior art, and

FIG. 4 shows a schematical circuit diagram of a bioelectrical impedance measuring apparatus of the prior art.

The drawing figures do not limit the present invention to the specific embodiments disclosed and described herein. The drawings are not necessarily to scale, emphasis instead being placed upon clearly illustrating the principles of the preferred embodiment.

### DETAILED DESCRIPTION OF THE PREFERRED EMBODIMENTS

The present invention is susceptible of embodiment in many different forms. While the drawings illustrate, and the specification describes, certain preferred embodiments of the invention, it is to be understood that such disclosure is by way of example only. There is no intent to limit the principles of the present invention to the particular disclosed embodiments.

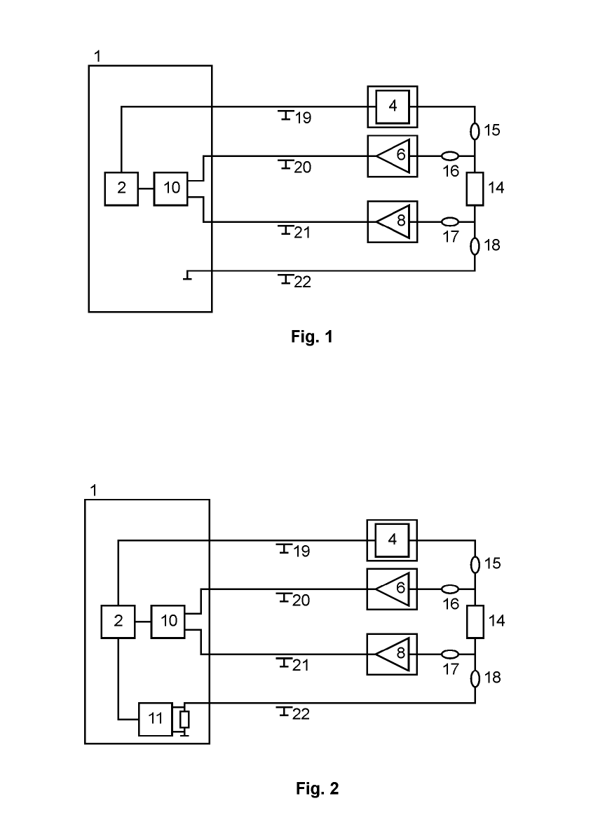

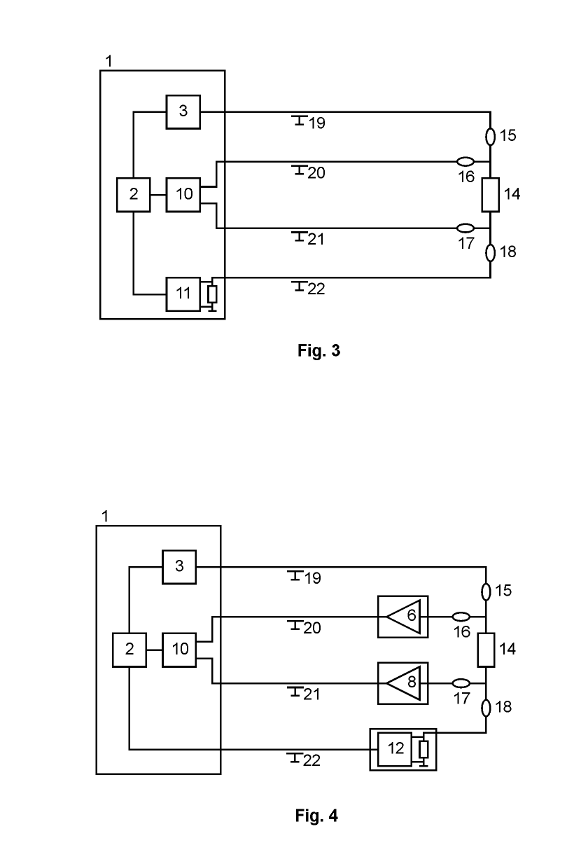

In the embodiments shown in FIGS. 1 and 2 of the present invention an alternating current source 4 by which an alternating current is applied via the electrodes 15 to the body is located as a separate component remotely from the main board 1 and in the vicinity of the applying electrode 15. In this manner it is impossible that the signal on the way from the main board to the remote electrodes at the limbs is subject to any corruption since the alternating current is generated by the alternating current source 4 in close vicinity to the electrode. The alternating current generated by the alternating current source 4 with a known amplitude predetermined by the control and analysis unit in this manner can not be deteriorated and is thus precisely known for the control and analysis unit 2. By measuring the voltage over the electrodes 16 and 17, the signals of which are passed on via preamplifiers 6 and 8 to the voltage measuring circuit 10, the voltage corresponding to the applied alternating current may be determined and the impedance may be derived therefrom. A separate measurement of the current is thus in principle no longer needed so that a current measuring circuit is omitted in the embodiment shown in FIG. 1.

In the embodiment shown in FIG. 2 a supplemental current measuring circuit 11 is provided on the main board to allow cross-checks.

When a good quality alternating current source is utilized measuring of the current may be obviated since the alternating current is sufficiently well known if the alternating current source is precisely controlled. In this manner the entire circuit component for current measurement may be saved and thus the bioelectrical impedance measuring apparatus may be simplified.

In addition, the amplitude of the applied alternating current can be reproduced in a more reliable manner in subsequent measurements since the amplitude of the applied alternating current in subsequent measurements can not be altered by changed cable paths in subsequent measurements.

The preferred forms of the invention described above are to be used as illustration only, and should not be utilized in a limiting sense in interpreting the scope of the present invention. Obvious modifications to the exemplary embodiments, as hereinabove set forth, could be readily made by those skilled in the art without departing from the spirit of the present invention.

The inventor hereby states his intent to rely on the Doctrine of Equivalents to determine and access the reasonably fair scope of the present invention as pertains to any apparatus not materially departing from but outside the literal scope of the invention set forth in the following claims.

What is claimed is:

1. Bioelectrical impedance measuring apparatus for determining composition data of a human body, said apparatus comprising:
a plurality of measuring electrodes;
a main board including measuring circuitry comprising voltage measuring circuitry and a control and analysis unit,
said control and analysis unit being configured to apply, according to a predetermined measuring program, an alternating current from a controllable alternating current source to an electrode specific for the respective measuring program to the body and to conduct away alternating current via another electrode,
said control and analysis unit being further configured to determine, based on two further electrodes and the voltage measuring circuitry, the resulting voltage, and to determine on this basis the impedance of a body segment,
each voltage applying electrode including a remotely controlled alternating current source separate from the main board and disposed in the vicinity of the current applying electrode.

2. Bioelectrical impedance measuring apparatus according to claim 1,
further comprising a preamplifier in the vicinity of the electrodes configured for voltage measurement for each electrode,
said preamplifier being separate from the main board.

3. Bioelectrical impedance measuring apparatus according to claim 1,
further comprising circuitry for measuring the applied alternating current.

4. Bioelectrical impedance measuring apparatus according to claim 1,
said electrodes being pairwise assigned to a respective one of the four limbs of a human body.

5. Bioelectrical impedance measuring apparatus according to claim 1,
said control and analysis unit generating a control signal for controlling the alternating current source as a differential signal transmitted on a twisted pair cable to the alternating current source.

6. Bioelectrical impedance measuring apparatus according to claim 1,
said control and analysis unit controlling the alternating current source using a signal transmitted to the alternating current source on a shielded cable.

7. Bioelectrical impedance measuring apparatus according to claim 2,
wherein voltage measuring signals from the preamplifiers located close to the electrodes are transmitted to the main board as differential signals on twisted pair cables to the control and analysis unit.

8. Bioelectrical impedance measuring apparatus according to claim 2,
wherein voltage measuring signals from the preamplifiers located close to the electrodes are transmitted to the main board on shielded cables to the control and analysis unit.

9. Bioelectrical impedance measuring apparatus according to claim 2,
wherein each electrode which conducts alternating current away from the body is connected to the main board via a shielded cable.
